(19) United States
(12) Patent Application Publication
(10) Pub. No.: US 2006/0030793 A1
(43) Pub. Date: Feb. 9, 2006

(54) Musculoskeletal dynamics assessment under weight-bearing conditions

(76) Inventors: Kevin P Granata, Blacksburg, VA (US); Steven M Zinder, Orange, CA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 10/539,814

(22) PCT Filed: Dec. 22, 2003
(86) PCT No.: PCT/US03/40670

§ 371 Date: Jun. 20, 2005

Related U.S. Application Data
(60) Provisional application No. 60/435,137, filed on Dec. 20, 2002.

(51) Int. Cl. A61B 5/103

(52) U.S. Cl. 600/592; 600/595

(57) ABSTRACT

In some illustrative embodiments, a method for the assessment of an ankle or the like includes: measuring musculoskeletal dynamics of an ankle or the like of a patient with the patient in a posture that is weight-bearing through the ankle or the like. In some embodiments, a device for assessing musculoskeletal dynamics of an ankle or the like of a patient in a weight-bearing posture can include: a platform to support a patient in a weight-bearing posture; a drive mechanism to impart pulse movement to the platform; a position sensor to sense a position of the platform; a force or torque sensor to sense a force or torque on the platform; a control system to determine musculoskeletal dynamics based on outputs from the displacement sensor and the force or torque sensor.

This application claims priority to U.S. Provisional Application Ser. No. 60/435,137 filed on Dec. 20, 2002, the entire disclosure of which is incorporated herein by reference.

### BACKGROUND

1. Field of the Invention

The present invention relates to analytical assessments of body parts. More specifically, the preferred embodiments relate to musculoskeletal dynamics assessments of body parts under weight-bearing conditions and, in some preferred embodiments, to musculoskeletal dynamics assessments of ankles under weight-bearing conditions.

2. Introduction

Ankle instability causes a wide variety of injuries including primary injury to the ankle (such as, e.g., strains, sprains, dislocations and/or the like) as well as secondary injuries related to falls (such as, e.g., injuries to knees, hips, arms, backs and/or heads) that occur as a result of unstable events at one's ankle(s). Often, individuals complain that they have “twisted an ankle.” In the United States, there are about 27,000 ankle injuries occurring each-and-every day. Moreover, more than about 1 million people visit emergency rooms and clinics with acute ankle sprains each each-and-every year. As for secondary injuries, the numbers, costs, severities and mortalities of secondary injuries associated with falls or the like initiated by ankle failures have not been quantified. However, falls have been identified as a significant health risk, such as, e.g., with respect to aging individuals.

The present inventors have found that there is a need to quantify the stability of ankles in order to, e.g., a) identify individuals at risk of injury, b) assess rehabilitation of individuals, c) evaluate prophylactic measures taken for individuals (such as, e.g., the bracing of an ankle, the taping of an ankle and/or the like) and/or d) develop improved treatments and/or diagnostic protocols.

While there are a variety of existing ankle assessment and/or the like systems, such systems have a variety of limitations. Among other limitations, some existing systems are used to evaluate ankle characteristics while the evaluated patient or test subject is in a non-weight-bearing posture. For example, some existing systems position the subject in a supine, or laying back position, or in a sitting position so as to avoid weight-bearing loads on the ankle. However, the present inventor's have determined that it is preferable to evaluate ankles or the like while the subject is in a weight-bearing position, such as, for example, in a standing position and/or the like. In this manner, among other things, the characteristics of the ankle can be, e.g., similar to that in a use condition (e.g., with the muscles in active state rather than a passive state) such as, e.g., when standing and/or the like.

In addition, while some existing systems have been designed to evaluate ankle flexibility, ankle laxity and/or the like relaxed-state characteristics and/or have been designed to assist in the exercise and/or rehabilitation of ankles, existing systems do not suggest, among other things, a) measuring ankle dynamics (such as, e.g., inertia, damping and/or stiffness) with a patient in a weight-bearing posture and/or b) taking similar measurements in an inversion/eversion plane of motion.

Thus, while a variety of systems and methods have been employed related to ankle analyses, the present inventors have found that there are number of limitations in existing systems and methods. Some illustrative systems and methods are illustrated in the following documents.

The Clinical Biomechanics article entitled Quantitative Measurement of Ankle Passive Flexibility Using an Arthrometer On Sprained Ankles (2001) involves the examination of “passive flexibility” of an ankle. The article explains that “[t]he human ankle joint is stabilized passively by restraints of ligaments, articulated surfaces and other connective soft tissues, and actively by muscle and tendon units. A decrease of passive stiffness of the joint has been suggested to represent a mechanical laxity indicating a weakness in one or several components of passive joint restraints. Joint flexibility is often used in practice for quantifying passive mechanical laxity of the ankle joint.” See Id. at 238. In the passive methods employed in this reference, “[d]uring the test, each subject sat upon a chair with his/her knee flexed at about 45° . . . ” and his/her “ankle . . . adjusted to its neutral position.” See p. 238. Among other things, the article does not suggest assessment in a weight-bearing condition and/or in a non-passive or muscle-active condition. Among other things, the document also does not suggest the assessment of joint dynamics, such as, e.g., inertia, damping and/or stiffness.

The Clinical Biomechanics article entitled Quantitative Assessment of Ankle Joint Dynamics During Recover From Injury (1990) involves the determination of “dynamic characteristics” of ankles in their passive state. As explained in the article, an “ankle was rotated passively from a neutral position to maximum dorsiflection and then to maximum plantarflexion.” See p. 188. In addition, “[s]ubjects lay supine with the left foot attached to the pedal.” Among other things, the article does not suggest assessment in a weight-bearing condition and/or in a non-passive or muscle-active condition. Among other things, the article also does not suggest rotation other than dorsiflexion and plantarflexion.

U.S. Pat. No. 6,162,189 entitled Ankle Rehabilitation System involves “a system for rehabilitating an ankle in which a mobile platform receives a patient's foot.” See Abstract. The '189 patent includes “exercises for balance, flexibility and strength.” Id. Among other things, the '189 patent does not suggest the assessment of joint dynamics, such as, e.g., inertia, damping and/or stiffness.

U.S. Pat. No. 5,402,800 entitled Ankle Laxity Measurement System involves a system for measuring laxity of an ankle. Among other things, the '800 patent does not involve the assessment of joint dynamics, such as, e.g., inertia, damping and/or stiffness. In addition, the '800 patent involves passive measurements rather than muscle-active measurements. See, e.g., column 4, lines 34+, “the unit is zeroed to make sure that the . . . load cells 34 and 35, are zero.” Among other things, the patent also does not suggest measuring in a weight-bearing condition and/or in a non-passive or muscle-active condition.

In addition, a variety of other known systems and devices are seen in, e.g., U.S. Pat. No. 6,277,057 entitled Ankle Rehabilitation Device, U.S. Pat. No. 5,810,703 entitled Exercise Board Having Central Mounting With Multi-Level Adjustable Spacer, U.S. Pat. No. 5,549,536 entitled Rotating Platform Apparatus, U.S. Pat. No. 5,766,119 also entitled Rotating Platform Apparatus, U.S. Pat. No. 4,199,137 entitled Apparatus for Foot Rehabilitation, U.S. Pat. No. 6,377,178 entitled Therapeutic Ankle & Foot Apparatus Having Contact Sensor Mechanism, European Patent Specification No. 0,324,279 entitled System for Measuring Fracture Stiffness, and U.S. Pat. No. 4,735,195 entitled Device Encouraging Periodic Joint Motion and Muscle Activity.

While a variety of systems and methods are known for the assessment and/or the like of body parts, such as, e.g., ankles, there remains a need for improved systems and methods for assessments of body parts, including, e.g., assessments of ankles.

### BRIEF DESCRIPTION OF THE DRAWINGS

The preferred embodiments of the present invention are shown by a way of example, and not limitation, in the accompanying figures, in which:

FIG. 1 is a perspective view of a first embodiment of the invention useful for, among other things, the assessment of a patient's ankle;

FIG. 2(A) is a schematic front view of a patient upon a device such as, e.g., that shown in FIG. 1;

FIG. 2(B) is another schematic front view of a patient upon a device such as, e.g., that shown in FIG. 1;

FIG. 2(C) is a schematic side view of another embodiment in which a weight-bearing load is applied using weights or the like;

FIG. 2(D) is a schematic side view of another embodiment in which a weight-bearing load is applied using a pulling mechanism or the like;

FIG. 3(A) is a schematic front view of another embodiment of the invention related to assessment of, for example, a patient's hip;

FIG. 3(B) is a schematic front view of another embodiment of the invention related to assessment of, for example, a patient's spine;

FIG. 3(C) is a schematic front view of another embodiment of the invention related to assessment of, for example, a patient's wrist;

FIG. 4(A) is a front view of a modification of the embodiment shown in FIG. 1;

FIG. 4(B) is a side view of the embodiment shown in FIG. 4(A);

FIG. 5(A) is a front view of another modification of the embodiment shown in FIG. 1;

FIG. 5(B) is a side view of another modification of the embodiment shown in FIG. 1; and

FIG. 5(C) is a schematic diagram demonstrating pulses applied to a motor shaft as an explanatory example.

### SUMMARY OF THE INVENTION

The preferred embodiments of the present invention can significantly improve upon existing methods and/or apparatuses.

According to some embodiments, a method for the assessment of a joint is provided that includes: measuring musculoskeletal dynamics of a joint of a patient with the patient in a posture that is weight-bearing through the joint. Preferably, the measuring musculoskeletal dynamics of a joint involves measuring musculoskeletal dynamics of an ankle, and, preferably, the measuring involves measuring musculoskeletal dynamics of an ankle in an inversion and/or eversion direction of rotation. In some embodiments, the measuring musculoskeletal dynamics includes measuring inertia, resistance and/or stiffness. In some preferred embodiments, the measuring musculoskeletal dynamics includes: applying at least one pulse so as to excite a natural frequency at the joint; and acquiring position and torque sensor data after initiating the at least one pulse.

According to some embodiments, a device for assessing musculoskeletal dynamics of a joint of a patient in a weight-bearing posture is provided that includes: a platform to support a patient in a weight-bearing posture; a drive mechanism to impart pulse movement to the platform; a position sensor to sense a position of the platform; a force or torque sensor to sense a force or torque on the platform; a control system to determine musculoskeletal dynamics based on outputs from the displacement sensor and the force or torque sensor. In some embodiments, the control system includes a computer programmed to determine values of inertia, resistance and stiffness based on data from the position sensor and the force or torque sensor.

According to some embodiments, a device for assessing musculoskeletal dynamics of a body part of a patient in a weight-bearing posture is provided that includes: a platform that supports a patient in a weight-bearing posture; the platform being rotatably supported to rotate around an axis through the body part; a drive mechanism that imparts a plurality of pulses to the platform at durations of less than about 50 milliseconds and at intervals of less than about 100 milliseconds; an angle sensor that senses an angular position of the platform; a torque sensor that senses a torque at the platform; digital data storage having angle and torque time-based data from the angle and torque sensors. In some preferred embodiments, the device is configured to assess musculoskeletal dynamics of a patient's ankle in a weight-bearing upright standing posture and with the ankle rotating in an inversion and/or eversion direction upon the platform. Preferably, the angle and torque time-based data obtained from the angle and torque sensors includes at least about 10 data points per second, or, in some embodiments, at least about 20 data points per second, or, in some embodiments, at least about 100 data points per second.

The above and/or other aspects, features and/or advantages of various embodiments will be further appreciated in view of the following description in conjunction with the accompanying figures. Various embodiments can include and/or exclude different aspects, features and/or advantages where applicable. In addition, various embodiments can combine one or more aspect or feature of other embodiments where applicable. The descriptions of aspects, features and/or advantages of particular embodiments should not be construed as limiting other embodiments or the claims.

### DETAILED DESCRIPTION OF THE PREFERRED EMBODIMENTS

While the present invention may be embodied in many different forms, a number of illustrative embodiments are described herein with the understanding that the present disclosure is to be considered as providing examples of the principles of the invention and such examples are not intended to limit the invention to preferred embodiments described herein and/or illustrated herein.

Musculoskeletal Stability

In preferred embodiments, musculoskeletal stability involves the potential of an articulating joint or the like to return to and/or remain in an appropriate anatomic alignment, such as, e.g., following a perturbation and/or mechanical disturbance. Musculoskeletal stability can relate to a variety of portions of an individual's body, such as, e.g., the individual's ankles, hips, knees, wrists, shoulders; vertebrae (e.g., lumbar region or elsewhere); and/or the like.

By way of example, when an individual walk's on loose or uneven ground surfaces, inappropriate foot placement and/or uneven foot or leg loading can generate mechanical forces and/or moments that may tend to roll one's ankle outward (e.g., eversion) or inward (e.g., inversion). This can result in pain and/or injury. To avoid this problem, active muscles crossing the leg-ankle-foot complex typically control the posture of the ankle to avoid unexpected rotation that may exceed injury tolerance. In addition, this control of the ankle posture also operates to maintain an upright posture of an individual.

There are a number of factors that contribute to the alignment stability of the ankle, including, e.g., visco-elastic dynamic properties of the ligaments, joint capsules and controlling muscles. In this regard, the active stiffness from the muscles plays a major role in stability. These factors are referred to herein as ankle dynamics parameters. These ankle dynamics parameters can include, e.g., a) inertia I, b) mechanical resistance to rotational velocity b (e.g., including differential neurophysiologic feedback or reflex), and rotational stiffness k (e.g., including proportional neurophysiologic feedback or reflex). The physiology of the ankle (or of another body part) changes as the load through the ankle (or of another body part) changes due to muscle activity and the like.

In view of the contribution of active muscles and/or the like in dynamics parameters, in the preferred embodiments of the invention, such dynamics parameters are assessed while a patient or test subject is in a weight-bearing position. In this application, the terminology weight-bearing posture includes postures in which a sufficient percentage of the subject's body weight is supported by the body part (e.g., ankle) such that muscles and/or the like of the body part are active due to the weight-bearing force applied. In some preferred embodiments, a weight-bearing position is selected such that muscles and/or the like at such body part are active in a manner to help stabilize and/or balance the subject, either substantially without external stabilization and/or balancing assistance (such as, e.g., using of braces, hand holds or the like to assist balancing the subject and/or using external stabilization or external balancing assistance in some embodiments). In some preferred embodiments, a weight-bearing position is selected such that vector directions of loads are similar to that during normal use, such as, e.g., in a direction of normal use while standing (e.g., for ankles) and/or the like.

In some preferred embodiments of the present invention, a musculoskeletal ankle dynamics device is provided. Preferably, the device includes a platform upon which a person stands while the device measures biomechanical dynamics (e.g., stiffness) of the ankle joint while the individual is in a weight bearing posture.

As a significant improvement over other forms of biomechanical evaluations of dynamic joint behavior, the preferred embodiments of the invention provide for measurements in weight-bearing postures. In addition, the preferred embodiments also preferably involve measurements in inversion and/or eversion directions. While some devices commonly known as “wobble boards” designed for therapeutic purposes may provide for motion in a number of directions, such are designed for therapy and are not designed to quantify, for example, biomechanical stiffness of the ankle.

As another significant improvement over, e.g., ankle laxity measurements and/or the like which involve situations in which the ankle is in a substantially relaxed condition (i.e., where muscles are substantially inactive), the preferred embodiments of the present invention allow for assessment of a patient while in a weight-bearing posture, such as, e.g., standing, in which muscles are in a substantially active state.

In the preferred embodiments, a musculoskeletal ankle dynamics device is used that employs engineering systems theory to quantify ankle dynamics, such as, e.g., ankle stiffness in functional weight-bearing postures. Because ankle injuries and subsequent falls frequently occur as a result of inversion and/or eversion disturbances, the preferred embodiments of the device are used to evaluate ankle performance in the coronal plane of motion (i.e., laterally side-to-side). In addition, in some embodiments, sagittal measurements (e.g., plantarflexion and/or dorsiflexion) can also be performed.

In preferred embodiments, in order to simulate the stability of the ankle during use (e.g., standing), the device is designed to evaluate ankle performance in upright, weight-bearing postures. In some preferred embodiments, the weight-bearing posture can include a posture applying about 50% of the test subject's body weight. In other embodiments, the percentage of the test subject's body weight can be at other values, such as, e.g., at about 20%, 30%, 40%, 60%, 70%, 80%, 90%, 100% and/or at about any other appropriate value between 0-100%. In addition, in other embodiments, weighted testing with loads of greater than 100% may be employed.

### Overview of the Preferred Embodiments

In some illustrative embodiments, small perturbation forces are applied to a subject's ankle, ankle movements are recorded, and ankle dynamics are estimated—including estimations of, e.g., a) neuro-mechanical resistance to ankle rotational angle (e.g., stiffness), b) resistance to ankle rotational velocity (e.g., damping), and/or c) ankle inertia. These parameters are referred to herein as musculoskeletal ankle dynamics parameters. As indicated above, these parameters are notable factors in ankle stability. In some embodiments, various other measurements, such as, e.g., measurements of angles of motion, extents of motion, rates of motion, frequencies of motion, etc., can also and/or alternatively be performed with devices according to some embodiments of the invention.

In preferred embodiments, a device can be provided that measures ankle dynamics in an inversion/eversion plane of motion and that can obtain data with the patient in a functional weight-bearing posture. In preferred embodiments, the device is designed such that the patient will stand with one foot on a device platform. In some preferred embodiments, the other foot will be supported on a fixed support (such as, e.g., a floor or solid ground). Preferably, the device can measure ankle dynamics parameters during the performance of a functional task (such as, e.g., standing upright) in a weight-bearing stance.

In preferred embodiments, when the user's foot is on the platform, an axis of rotation of the cradle is approximately aligned with an axis of rotation of the patient's ankle (such as, e.g., at an anatomic elevation of the lateral maleoli). As described above, because ankle injuries and subsequent falls frequently occur as a result of inversion or eversion disturbances, the device is preferably configured to enable evaluation of ankle performance in the coronal plane of motion (e.g., laterally side-to-side). As also described above, other measurements, such as, e.g., sagittal (e.g., plantarflexion and/or dorsiflexion) measurements can also be performed in some embodiments. By way of example, in some embodiments, the same device can be used to take various measurements. For example, in some embodiments, when a patient's foot is placed longitudinally along an axis of rotation of a platform, then rotation of the platform can cause ankle inversion and/or eversion. On the other hand, when a patient's foot is placed laterally (e.g., transverse to the axis of rotation of the platform), then rotation of the platform can cause ankle plantarflexion and/or dorsiflexion. In some embodiments, a patient's foot can be placed and oriented at any desired angle, such as, e.g., at any angle between the above two orientations to produced, e.g., a biplanar motion.

In some preferred embodiments, a drive mechanism, such as, e.g., a motor is used to impart small angular movements to the cradle. In this manner, the drive mechanism will cause small angular movements about the patient's ankle. In various preferred embodiments, the drive mechanism can be controlled by a control system. In some preferred embodiments, the control system can include software, firmware, hardware and/or the like and may include, e.g., a programmable computer, a programmable logic controller, a processor and/or any other appropriate control device(s) now or later known to those in the control systems arts. In some preferred embodiments, the control systems can be used to cause the drive mechanism to generate a time-sequence train of periodic and/or non-periodic ankle movements. Preferably, these ankle movements will result in an applied torque that can be recorded (such as, e.g., based on sensor outputs) by the device. In this regard, in preferred embodiments, sensors are provided that sense the position of a support platform and that sense forces applied to the platform by the body part (e.g., ankle) of the patient. While in some preferred embodiments described herein the movement of the ankle, joint or the like body part can be inferred based on movement of a platform or device, in some other embodiments various other methods of measuring and/or recording movement of the ankle, joint or other body part of interest can be employed. For example, the motion can be recorded by any now or later known motion detecting means(s), which may use physical sensors, optical sensors and/or the like. In some examples, commercially available electrogoniometers (such as, e.g., in some illustrative examples those using a potentiometer that gives a varying output voltage dependent upon an angle between two bars attached to respective body portions and/or other mechanisms) can be used to record motion of the ankle, joint or the like directly instead of inferring the movement based upon, e.g., movement of the platform and/or drive mechanism motion.

While the most preferred embodiments employ a motor, a motor could be eliminated in some less preferred embodiments. For example, in some embodiments, movement could involve a patient's ankle reflexes during maintenance of postural stance and/or the like and/or another external source could impart movement.

In some preferred embodiments, the recorded data can be inputted into a analysis control system, which can include, e.g., a computer, a processor and/or the like (which can be, e.g., the same control system described above or an independent control system) and used to determine or estimate ankle dynamics parameters. In this regard, in some embodiments, the analysis control system can include, e.g., a program module having analysis software in which dynamics parameters can be determined. For example, the analysis software can be programmed to determine the dynamics parameters in some embodiments from a second-order (or higher) parametric model. In preferred embodiments, outputs of the analysis software can include, e.g., ankle stiffness, damping and/or inertia of the ankle associated with ankle joint and postural stability. In the preferred embodiments, the device can be used to provide quick, non-invasive, quantitative assessment of parameters (e.g., ankle stability parameters).

In some illustrative and non-limiting examples, one or more of the following illustrative services can be performed using some exemplary devices according to the present invention (NB: various embodiments are not limited to these illustrative services, but may involve some of these and/or other services):
1) Assessment of ankle stability for clinical evaluation of:
   a) Patients at high risk of slips, trips and/or falls;
   b) Post injury of ankle dysfunction; and/or
   c) Prospective identification of athletes or other individuals at risk of potential ankle injury.
2) Assessment of rehabilitation status and/or progress:
   a) Quantitative documentation of status for rehabilitation baseline assessment; and/or
   b) Quantitative documentation of status for rehabilitation follow-up assessment.

In some of the preferred embodiments of the invention, one or more potential advantages can be obtained (NB: various embodiments are not limited to these illustrative advantages but may achieve some of these and/or other advantages):
1. In vivo musculoskeletal ankle dynamics parameters can be quantified (such as, e.g., using engineering systems theory).
2. The assessment approach can be quick and non-invasive, with musculoskeletal ankle dynamics parameters provided substantially immediately upon completion of the test.
3. The assessment can be performed with the patient in an upright, weight-bearing posture
4. The assessment permits evaluation of stability parameters in the inversion/eversion plane of ankle movement (i.e., the movement direction typically associated with ankle instability, sprain and/or injury).

### Detailed Description of the Illustrated Embodiments

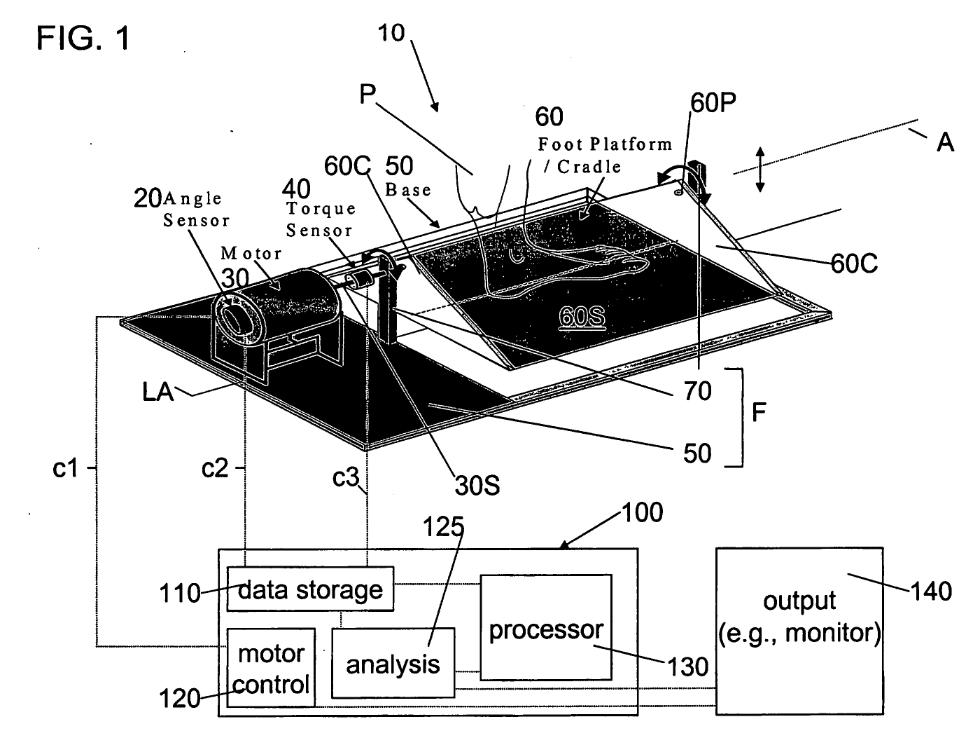

While the present invention may be embodied in many different forms (as indicated above), FIG. 1 illustrates some exemplary features that can be employed in some preferred embodiments related to a musculoskeletal ankle and/or the like dynamics assessment device that can include, e.g.: 1) a swinging platform or cradle 60 for foot placement; 2) a support frame F for supporting the cradle 60; 3) a motor 30; 4) a force sensor 40 to record the applied force (e.g., torque=force×distance); 5) an angle sensor 20 to record the cradle angle; and 6) at least one control system(s) 100 (to, e.g., provide motor control, parametric analyses [such as, e.g., to compute stiffness] from acquired data, and/or other functionality).

As shown in FIG. 1, the device 10 is constructed such that a patient P can stand with one foot on a support platform or cradle 60 (i.e., in a weight-bearing posture). In preferred embodiments, the motor 30 is used to cause small inversion and/or eversion movements about the ankle. In the illustrated embodiment, the position is sensed using at least one angle sensor(s) 20 that sense the angular orientation of a motor drive shaft 30S (i.e., which corresponds to the orientation of the platform 60). In addition, in the illustrated embodiment, the forces applied are sensed using at least one torque sensor(s) 40 that senses the applied torque on the shaft 30S. In preferred embodiments, data received from the sensors is stored for evaluation of the movement dynamics and parametric identification software is used to determine and output values related to ankle inertia, damping and/or stiffness.

The motor 30 is preferably used to impart small angular movements to the cradle, while the sensors 20 and 40 are preferably used to record the movement of the foot as well as the forces necessary to move the foot. In the illustrated embodiment, the cradle 60 includes a foot platform and a pair of upright walls 60C that are supported via columns 70 so as to pivot around the axis A extending through a center line of the shaft 30S. The shaft 30S extends through the column 70 and is rotatably supported therein (such as, e.g., via rotational bearings) and is fixedly connected to a wall 60C of the platform so as to move in unison therewith. The opposite wall 60C is preferably pivotally supported at 60P. In this manner, the platform 60 can swing about the pivot axis A along with the angular movement of the shaft 30S. In some preferred embodiments, the foot-platform 60 is formed with a substantially flat upper surface 60S, and, in some embodiments, the platform can have a substantial flat and rectangular configuration as shown. Preferably, the foot platform 60 is substantially symmetrical about the longitudinal axis LA and is substantially lightweight so as to have a limited impact on the motion of the device.

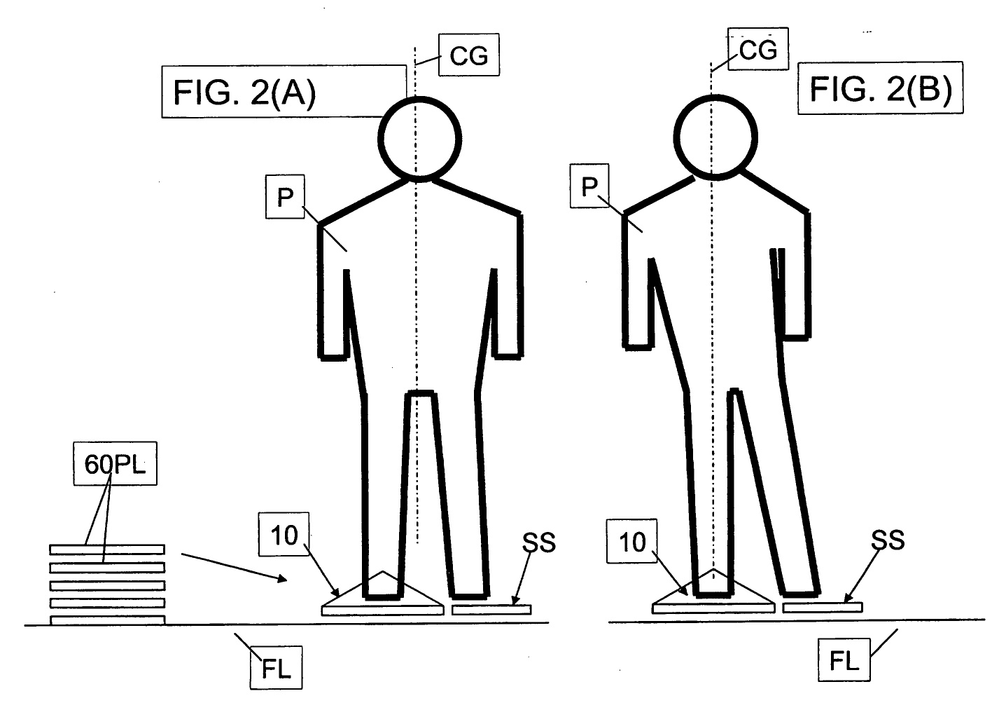

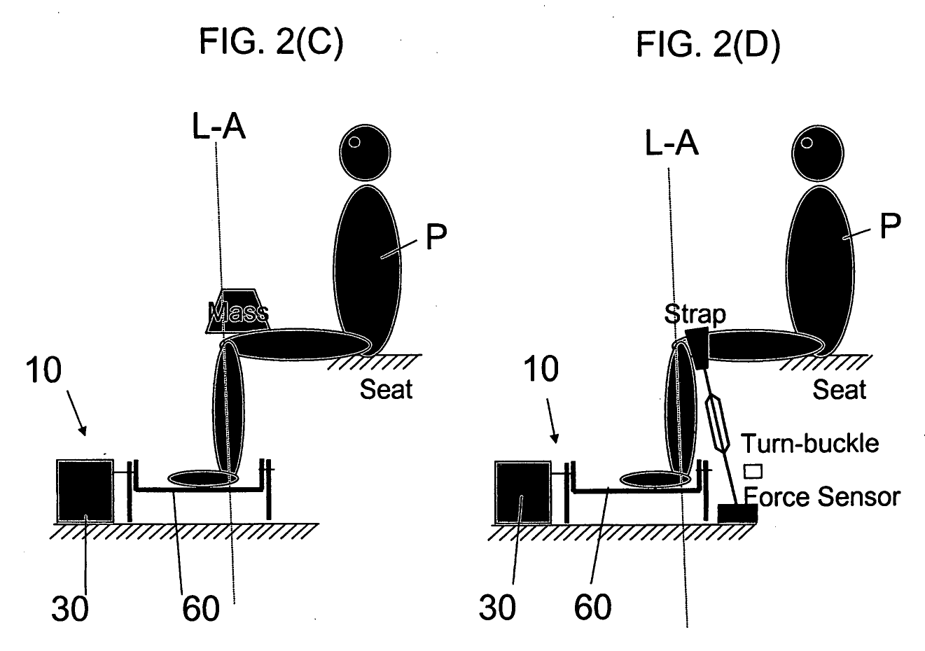

In preferred embodiments, the columns 70 are designed to support the foot-platform 60 at an elevation beneath the elevation of a pivot axis. Preferably, the columns 70 are configured to suspend the foot-platform 60 below a pivot axis A that extends through a desired location of the user's foot (as discussed above). Preferably, when the patient P is standing with one foot on the cradle 60, the axis of rotation A of the cradle is approximately aligned with the axis of rotation of the ankle, such as, e.g., at the anatomic elevation of the lateral maleoli. In some embodiments, the relative positions between the two horizontally aligned axles that support the platform walls 60C and the surface 60S of the platform is adjustable in order to facilitate proper alignment for use with varied patient configurations. In this regard, in some embodiments, the walls 60C can be made so as to be extendable (such as, e.g., using telescoping members and/or the like). In other embodiments, the platform 60S can be raised and/or lowered as desired. By way of example, in some simplified constructions, in order to accommodate different sizes, additional plates 60PL, shown in FIG. 2(A), can be provided that can be placed underneath a patient's foot to adjust the height accordingly (i.e., based on the number of plates used, with the maximum height employing zero plates).

In the example shown in FIG. 1, the two separate horizontally aligned axles or pivots support the respective walls 60C to enable the cradle to swing upon the frame F, while allowing the patient P to freely place his or her foot on the foot-platform of the cradle. While in the illustrated example, the frame F includes, e.g., a base 50 and columns 70, a variety of other frames or supporting structures could be employed in various other embodiments. With the structure shown in FIG. 1, when a patient's foot is placed longitudinally on the platform (such as, e.g., shown in FIG. 1), then the rotation of the cradle can result in ankle inversion and/or eversion. In addition, with this structure, when the patient's foot is placed laterally upon the platform, then rotation of the cradle can result in ankle plantarflexion and/or dorsiflexion. In addition, with this structure, the patient's foot can be positioned at any desired angle in between these longitudinal and lateral orientations to produce a biplanar motion.

In the embodiment shown in FIG. 1, the support frame F is constructed with a large base 50 that is sufficient to ensure stability of the device and to fixedly support the columns 70 at their lower ends. The support columns 70 preferably support the cradle at an elevation sufficient to ensure free movement of the cradle such that it does not abut the ground or another surface when it is caused to rotate (i.e., at least within a desired range of motion). The support frame is preferably of a sufficient strength to safely permit a patient's full body weight to be applied to the cradle while it hangs from the support columns 70.

In the embodiment shown in FIG. 1, the motor 30 is preferably fixedly attached to the base 50 of the support frame F. In some preferred embodiments, the motor is an electric motor. However, any appropriate motor and/or other form of drive mechanism, such as, e.g., a solenoid, a hydraulic cylinder and/or any other drive mechanism can be used in other embodiments. As described above, the drive shaft or axle 30S of the motor is rigidly attached to the cradle (i.e., directly or indirectly via one or more intermediate elements) and causes the cradle 60 to rotate about the axis A.

As shown in FIG. 1, a control system 100 is preferably provided which can include, e.g., one or more computer(s) and/or one or more network(s) of computer(s), one or more programmable logic controller(s) and/or any other appropriate control devices. Illustrative computers can include, e.g.: a central processing unit; memory (e.g., RAM, etc.); digital data storage (e.g., hard drives, etc.); input/output ports (e.g., parallel and/or serial ports, etc.); data entry devices (e.g., key boards, etc.); output devices (e.g., monitors, printers, etc.). In some illustrative embodiments, motor control and data collection can be performed using LABVIEW software by NATIONAL INSTRUMENTS. In some illustrative embodiments, other functionality, including analysis functions, can be performed using, among other things, MATLAB software by THE MATHWORKS. Additionally, various other software programming languages and/or methods can be employed in various implementations. By way of example, in some embodiments, controller software written in other programming languages such as C++ and/or Assembler Language can be used to, e.g., run on microprocessors. Similarly, in some embodiments, analysis functions can be written in C++ and/or other appropriate programming lanuages. It should be understood that the various forms of programming are merely illustrative examples and in various embodiments any appropriate form of programming, coding and/or the like can be employed. In some embodiments, a motor control module 120 can be used (such as, e.g., programmed into a computer, hardwired into a programmable logic controller and/or the like) to control the motor to induce small movements of the cradle 160. As described above, the motor 30 preferably causes the cradle to perform small angular movements about a generally horizontal axis A so as to cause, for example, the patient's foot to rotate within an ankle inversion and/or eversion plane of movement. Once again, depending upon the patient's foot placement, the patient's foot can also be made to undergo small angular movement in a plantarflexion and/or dorsiflexion plane of movement and/or to undergo a biplanar motion. In preferred embodiments, the motor imparts these movements within a limited range of motion to avoid over-rotation of the patient's ankle and/or to avoid injury.

In some preferred embodiments, the control system 100 is programmed to cause angular rotation of the cradle 60 in a) pre-specified movement patterns and b) pre-specified angular movement amplitudes. In some preferred embodiments, the control system 100 can be programmed to control the motor 30 to rotate the cradle back and forth in a substantially periodic manner, such as, e.g. with a pre-set frequency of motion, with multiple-frequency sequences, with specified motion amplitudes and/or with specified durations of ongoing movement. In some preferred embodiments, the control system 100 can alternatively be programmed to control the motor to have any sequence of Fourier combination of sinusoidal movement. In some preferred embodiments, the control system can be programmed to control the motor to follow pseudo-random movement sequences (e.g., which can be pre-programmed). In some preferred embodiments, the control system 100 can control the motor to apply a baseline clockwise or counter-clockwise torque to the cradle, e.g., a DC or zero-frequency Fourier component of applied torque. In various embodiments, the motor and motor controller can be operated using either an open-loop control or a closed loop control.

In some embodiments, a transient control can also or can alternatively be implemented wherein the motor applies a brief force pulse and then allows the cradle to swing freely. Among other things, this can permit recording of the natural motion of the cradle and ankle following an angle or force perturbation. In some of the preferred embodiments, the device can operate in either a transient control mode (i.e., where substantially no motor torque is applied to the cradle) and/or in an active control mode (i.e., where the motor actively causes movement to the cradle). In the preferred embodiments, at least one angle sensor(s) 20 and at least one torque sensor(s) 40 are implemented to record movement and applied torque in either of these modes of operation.

In the embodiment shown in FIG. 1, the torque applied by the motor 30 to the cradle 60 is recorded by the torque sensor 40. In addition, the angle sensor 20 can include, e.g., encoders and/or rotational potentiometers that are used to record the angle of the shaft 30S (or to record the cradle angle and/or the like). This data can then be recorded at a predetermined sampling rate or the like and stored (e.g., in digital data storage 110) by the control system 100. In addition, in some embodiments, the sensor data can be displayed (such as, e.g., for biofeedback for the patients) during the taking of the measurements. In addition, in some embodiments, a weight-bearing load applied upon the cradle 60 can concurrently be recorded and/or displayed. In this regard, in order to determine the weight-bearing load, the device 10 can be supported on a scale or weight measuring unit (e.g., supporting the frame F), or a scale or weight measuring unit can be located on top of the platform between the user's foot and the surface 60S and/or the user's other foot can be located upon a scale or weight measuring unit, such that a weight-bearing load applied to the device 10 can be discerned. While a weight-bearing load may be selected based on circumstances, it should be appreciated that in the preferred embodiments, the weight-bearing load is applied toward the platform substantially centered at and/or along the longitudinal axis LA in a manner so as to have a minimal impact on the motion of the platform 60.

As shown in FIG. 1, the control system 100 can include an analysis module 125 (e.g., which can include software for carrying out parametric analyses). The analysis module can carry out parametric analysis based on musculoskeletal ankle dynamics from the measured torque values obtained by the torque sensor and the measured position values obtained by the angle sensor (e.g., which can be stored in digital data storage 110).

In the analysis module 125, in some preferred embodiments, the data obtained from the position and force sensors (which data is recorded as time-domain sensor data) can be fitted to a second-order model of motion as follows:
{Is²+bs+k}Θ(t)=T(t) (1)
where I is the inertia (e.g., of the cradle and patient's foot), b is the mechanical resistance to rotational velocity including differential neurophysiologic feedback or reflex, and k is the rotational stiffness including proportional neurophysiologic feedback or reflex. In equation 1, Θ(t) is the angle of rotation of the cradle at each sampled time point t and T is the torque at each sampled time point t. The term s is the Laplace transform coefficient (such as, e.g., defined in common elementary engineering control theory textbooks). The coefficients I, b and k can be assumed to be slowly varying and a function of muscle activation due to body weight supported by the cradle.

In addition to equation 1, the same behavior can be expressed in a frequency domain as follows:
{Iω²+ibω+k}Θ(ω)=T(ω) (2)
where w represents the frequency component determined by conventional means and i={−1}^{1/2}.

In preferred embodiments, in the analysis module 125, the musculoskeletal ankle dynamics parameters (e.g., I, b and/or k) can be determined by fitting the measured data Θ and T to these second-order models. Higher-order models and time-delay methods can also be applied to estimate these coefficients (e.g., I, b and k). An output from the analysis module can, thus, include one or more, preferably all, of the effective ankle inertia I, the effective mechanical damping b and/or the effective stiffness k. The term “effective stiffness” and the like is employed because this device can measure the influence of, for example, the ligaments, the joint capsules, the passive muscle behavior, the active (e.g., intrinsic) muscle behavior, and the neuromuscular response or reflex behavior of the patient's system as a whole. These are primary components of the musculoskeletal stability of an ankle.

In some preferred embodiments, the analyses module 125 can, thus, be used to carry out, among other things, two major steps: a) an initial step in which fundamental frequencies are isolated (such as, e.g., using various methodologies, such as, e.g., Fourier transforms, time domain deconvolution techniques, filtering techniques and/or any other appropriate methods); and b) the values of the dynamic parameters are determined as set forth above.

In some illustrative and non-limiting examples, an illustrative motor 30 that can be employed in some embodiments can be a PACIFIC SCIENTIFIC brushless electric servomotor Model PMA42M having a servo drive model PC8x2, SC9x2/SCE9x2, with a peak stall torque of about 7.7 Nm, a peak rated torque of about 7.6 Nm, a continuous stall torque of about 4.1 Nm, and having a motor feedback output including an angle position sensor output, wherein the angle sensor output has a sensitivity range of about 10,000 data points per revolution. In some illustrative and non-limiting examples, an illustrative torque cell that can be employed in some embodiments is an OMEGA torque cell model TQ301.

In some illustrative and non-limiting examples, the motor 30 can be activated for pulses of between about 5 to 50 milliseconds, or, in some embodiments, between about 10 to 40 milliseconds, or, in some embodiments, between about 15 to 30 milliseconds, or, in some embodiments, at a number of pulses having a mean value of generally about 20 milliseconds. Preferably, the applied pulses are imparted at short durations to enable excitation of the body part (e.g., ankle) over a wider range of frequencies. In this regard, a longer pulse would potentially introduce a particular frequency (e.g., a larger pulse may have a tendency to essentially push or swing the ankle at a particular frequency). In the preferred embodiments, the pulses are used in an effort to excite the body part's natural frequency. Typically, for an ankle, the natural frequency can be in a range of about 3-5 Hertz.

In some preferred embodiments, the pulses are imparted before a patient's voluntary control can take place because this voluntary control could affect the results. As a result, pulses are preferably at intervals of less than about 120 milliseconds (e.g., between starting times of pulses), or, in more preferred embodiments, at intervals of less than about 100 milliseconds, or, in some embodiments, within an interval range of about 50 to 100 milliseconds, or, in some embodiments, within an interval range of about 10 to 50 milliseconds. In some preferred embodiments, as described above, the pulses are imparted in a substantially random manner, such as, e.g., using a pseudorandom movement sequence.

Preferably, the device is adapted so as to limit the range of motion of the assessed body part during the collection of assessment data. In some preferred embodiments related to, e.g., the assessment of an ankle or another body part, the extent of angular motion is within a range of about plus-or-minus 15 degrees, or, in some embodiments, within a range of about plus-or-minus 10 degrees, or, in some embodiments, within a range of about plus-or-minus 5 degrees, or, in some embodiments, within a range of about plus-or-minus 2 degrees or even less. In some preferred embodiments, the allowed range of motion can be preset prior to operation.

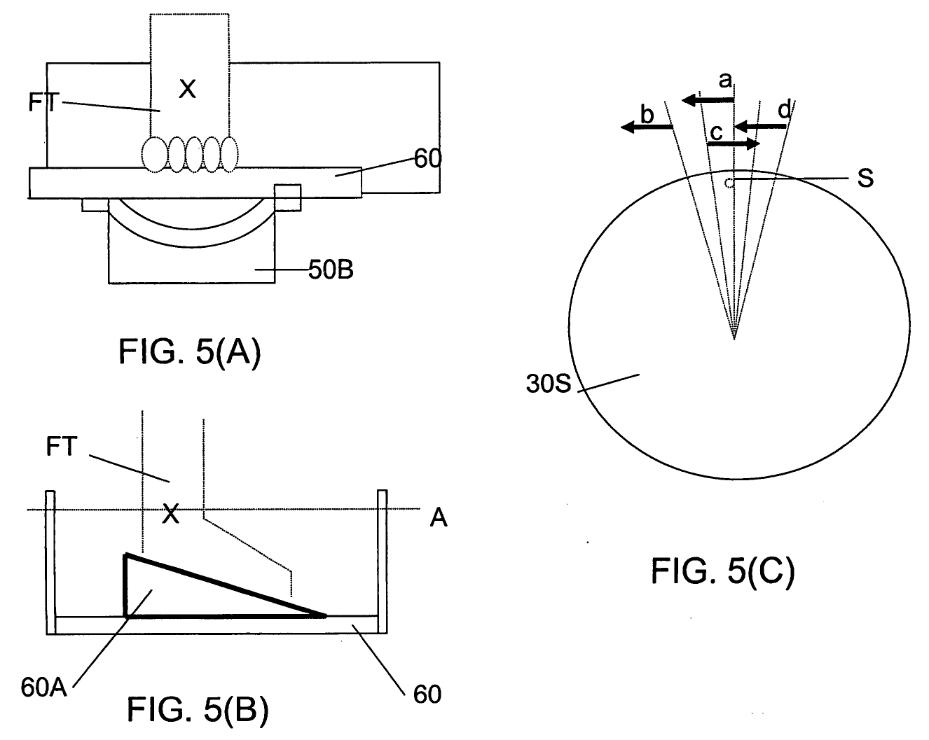

FIG. 5(C) is a schematic diagram illustrating that in various embodiments and/or during actual use of various embodiments, the pulses generated can be applied while the output shaft 30S of the motor is in a variety of orientations within a preset range (e.g., plus-or-minus 10 degrees or the like). In this explanatory example, four positions a, b, c and d corresponding to the first four pulses are shown. For explanatory purposes, a fictional point S on the shaft 30S will be rotated to the respective dotted lines shown corresponding to these positions in this non-limiting example. Here, the pulse a is an initial pulse imparted in a direction of the arrow adjacent thereto, the subsequent pulse b is similarly imparted in the same direction but after the shaft has been rotated to the position b, the subsequent pulse c is now imparted in the opposite direction but after the shaft has been rotated to the position c, and the subsequent pulse d is imparted in the original direction but after the shaft has been rotated to the position d. These points are used merely for illustrative purposes to demonstrate the pseudorandom nature that can be used in some embodiments.

In some preferred embodiments, data collection is performed at a rate of at least about 10 times per second (i.e., at least about 10 Hertz), or, more preferably, at a rate of at least about 20 times per second, or, more preferably, at a rate of at least about 50 times per second, or, more preferably, at a rate of at least about 100 times per second, or, more preferably, at a rate of at least about 200 times per second. In some embodiments, employing motors and sensors of PACIFIC SCIENTIFIC and OMEGA, as described above, angle sensor and torque sensor data collection rates can each be at about 250 times per second.

In some illustrative embodiments, the device can be operated to collect data for less than about one minute, or, in some other embodiments, for about 5 to 30 seconds, or, in some other embodiments, for about 10 to 20 seconds. In some illustrative embodiments, data can be collected over a time period corresponding to between about 25 to 300 pulses. In some other illustrative embodiments, data can be collected over a time period corresponding to about 50 to 150 pulses. While a substantial number of pulses is desired in some preferred embodiments, in some embodiments the device could collect data following just a single pulse, or, for a period of time over just a few pulses.

While in the most preferred embodiments torque sensors are employed, in some embodiments torque sensors could be omitted. In this regard, in some embodiments where torque sensors are omitted, stiffness calculations could be calculated based on motion characteristics detected using at least one angle or position sensor(s). However, because, among other things, muscles and the like will likely disturb the motion obtained, using torque sensors is preferred.

FIG. 2(A) illustrates an embodiment in which a patient P is standing in a substantially erect position with a center of gravity CG passing generally along the middle of the patient, such as, e.g., to distribute the patient's body weight substantially evenly between the patient's feet, upon the device 10 and upon the support surface SS. The support surface SS can include a support that is fixedly mounted with respect to a floor or ground FL, a weight-measuring device, another device 10 for similar analyses of the other ankle, and/or another appropriate surface for supporting the patient P.

FIG. 2(B) illustrates another embodiment in which a patient P is standing in a leaning position so as to impart more or substantially all of his or her body weight upon the device 10. FIG. 2(B) helps to demonstrate that the degree of body weight applied to the device 10 can be selected as desired, such as, e.g., by positioning of the patient and/or by other means, such as, e.g., providing partial support via support braces (not shown) for the patient, hand holding members (not shown), and/or the like. In the embodiment shown in FIG. 2(B), the center of gravity CG is shown as passing substantially through a center of the device 10. While the embodiment of FIG. 2(B) increases the body weight imparted upon the device 10 compared to that shown in FIG. 2(A), in other embodiments, the body weight upon the device 10 can be reduced from that shown in FIG. 2(A).

FIGS. 2(C)-2(D) show a few other means that can be employed for applying a weight-bearing load in some embodiments. For example, FIG. 2(A) shows an embodiment in which a weight-bearing load is applied using other means, such as, e.g., a weight MASS as shown. In some preferred embodiments, as shown in FIG. 2(A), the foot and lower-leg are preferably aligned in a position that is substantially identical to that in a standing configuration (such as, e.g., with the lower-leg along a generally upright, slightly forwardly tilted, axis L-A), but with the patient P's knees bent. Here, the patient P can be in a sitting position as shown. As shown, to apply an appropriate weight-bearing load through the foot and ankle, a downward force is preferably applied. By way of example, as shown, a weight MASS (such as, e.g., weight plates and/or any other appropriate weights) can be applied upon the patient's upper legs and/or knees. In some embodiments, the amount of weight applied can be selected based upon the weight of the patient P and/or based upon activities performed by the patient P (such as, e.g., to approximate certain normal use conditions). FIG. 2(B) shows another means in which a tension applying strap is used to provide a pulling force on a user's legs so as to achieve a similar result to that shown in FIG. 2(A). In this illustrative embodiment, a turn-buckle and/or other mechanism can used to apply such a pulling force as shown in FIG. 2(B). In this regard, a turn-buckle or the like may be located between a knee strap and a fixed support. In some embodiments, a force meter or force cell can be used. Preferably, the force meter or force cell can measure, record and/or display the applied downward load. While FIGS. 2(C) and 2(D) show some illustrative means for simulating standing, various other means for applying loads or forces similar to that in standing and/or the like weight-bearing postures can be employed in various other embodiments.

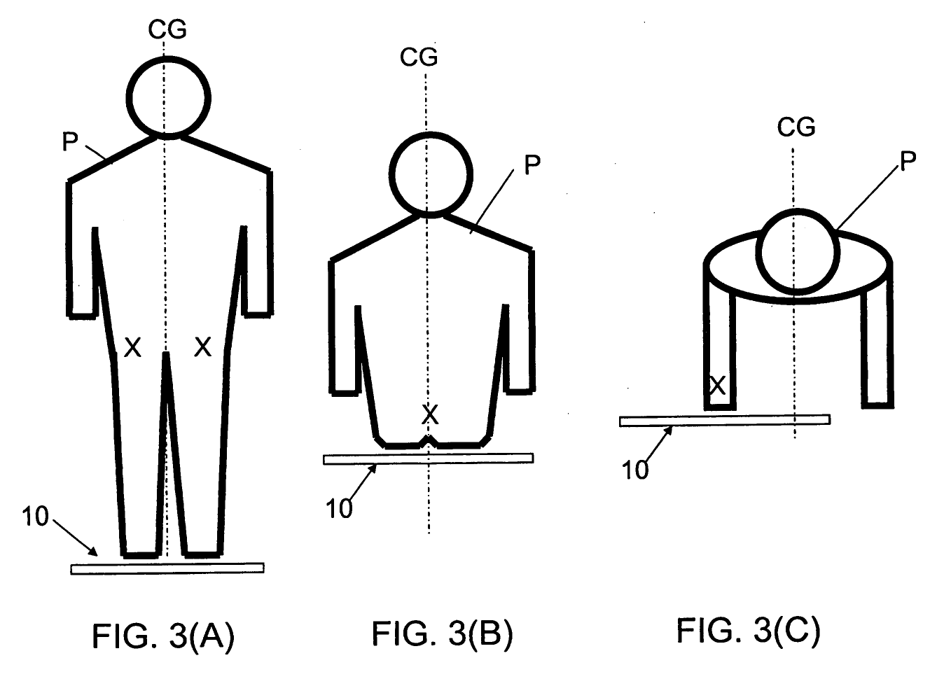

FIGS. 3(A)-3(C) show other embodiments that can be employed for, e.g., assessing other body parts. In this regard, in various embodiments, principles described herein can be used for the assessment of musculoskeletal stability and/or the like of various other body parts. By way of example, FIG. 3(A) shows an illustrative arrangement used for the assessment of a patient's hips (e.g., at locations X). In this illustrative embodiment, a user may stand on a device 10 with both feet as shown. In this example, the platform could potentially merely move side-to-side on a fixed plane, especially since the radial distance from the patient's hips to the patient's feet is substantially longer. As another example, FIG. 3(B) shows an illustrative arrangement used for the assessment of a patient's lower back (e.g., at location X), such as, e.g., for the assessment of the clinical stability of a spine. In this illustrative embodiment, a user may sit on a device 10 with his or her buttocks upon a platform or the like as shown. As another example, FIG. 3(C) shows an illustrative arrangement used for the assessment of a patient's wrist (e.g., at location X). In this illustrative embodiment, a user may, e.g., lean forward with one hand supported on a device 10 (e.g., in a crawling position) as shown. In some implementations of various embodiments, in order to isolate specific regions for assessment and/or evaluation, other portions of the body may potentially be wrapped, braced, bound and/or otherwise restricted from movement. In the embodiments shown in FIGS. 3(A)-3(C), as with the embodiment shown in FIG. 1, the movement of a platform of the device 10 is preferably imparted in such a manner as to cause a natural motion at the region(s) X shown in the respective figures. In this regard, for example, in FIG. 3(A), the platform of the device can essentially move parallel to a substantially planar path passing through the respective Xs shown. With respect to FIG. 3(B), the platform of the device can be made to rotate around an axis passing through the location X (which can, e.g., be selected to isolate a particular region). Similarly, with respect to FIG. 3(C), the platform of the device can be made to rotate around an axis passing through the location X.

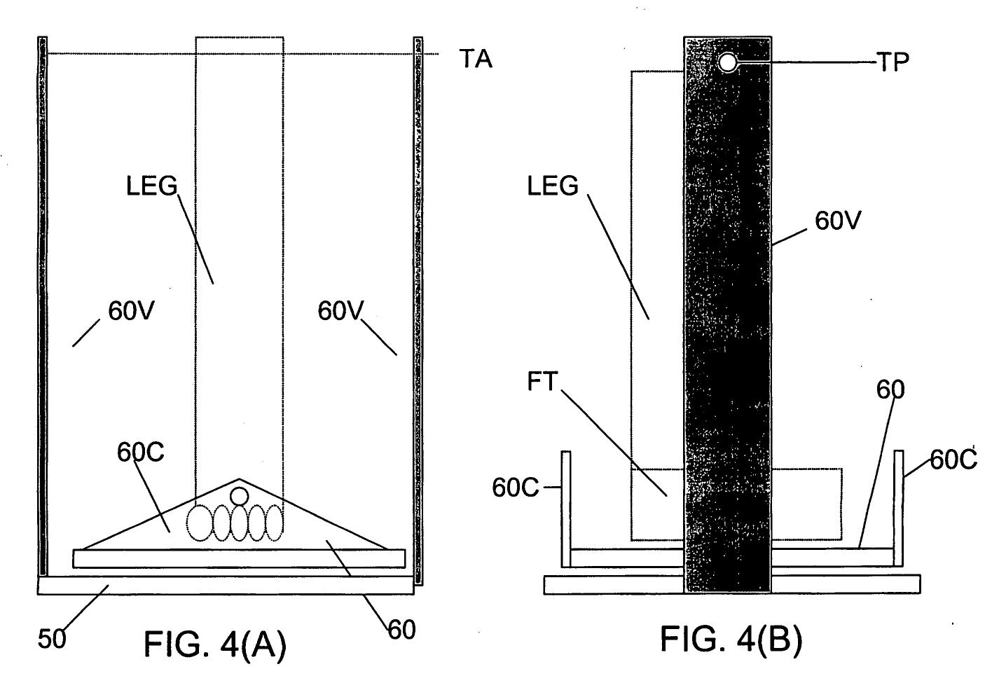

FIGS. 4(A)4(B) show another embodiment in which a device similar to that shown in FIG. 1 can potentially be used in an environment in which the weight-bearing load and/or angular position of the patient's leg LEG and foot FT may vary during the assessment period (such as, e.g., in a manner generally simulating walking). In this regard, FIG. 4(A) is an end view of a device similar to that seen from the right side of FIG. 1 along the axis A according to a modified embodiment in which the entire base 50 is supported via vertical supports 60V so as to pivot about a pivot point TP. Here, the pivot TP is preferably aligned with a patient's hip(s) so as to enable the device to swing along with a fore-to-aft swing of the patient's leg LEG. FIG. 4(B) is a left side view of the device shown in FIG. 4(A). In some embodiments, a similar swinging support surface (not shown) could be used to support the other foot (not shown), such that both legs can swing in an alternating manner (e.g., generally similar to walking).

FIG. 5(A) shows another embodiment in which a device similar to that shown in FIG. 1 includes a platform 60 that is supported to rotate upon a curved support 50B fixed to a base 50 (see, e.g., base 50 shown in FIG. 1). This embodiment demonstrates that the manner in which the platform is supported for movement with respect to the base 50 can be varied. In the embodiment shown in FIG. 5(A), bearings can be used to facilitate free movement of the platform on the support 50B. Here, appropriate angle and force sensors could be employed as would be understood based on this disclosure.

FIG. 5(B) is a side view of a platform 60 similar to that shown in FIG. 1 having an adapter member 60A placed on the top surface 60S so that a patient's foot FT assumes a toe down (e.g., plantarflexion) position during assessment of movement in the inversion and/or eversion directions. In this manner, for example, one or more adapter(s) 60A can be selected to conduct assessments under desired conditions. For example, the embodiment shown in FIG. 5(B) can be used to evaluate conditions such as, e.g., related to the use of high-heeled shoes. As shown in FIG. 5(B), the axis A is preferably still positioned so as to pass through the ankle location X, similarly to the embodiment shown in FIG. 1. While FIG. 5(B) includes an adapter member 60A, it is contemplated that the surface of the platform 60 could also be configured to assume a desired shape, angle and/or contour as desired based on circumstances. However, in some preferred embodiments, such as, e.g., shown in FIG. 1, the surface will be a substantially horizontal and planar surface to simulate a common floor or ground surface.

### BROAD SCOPE OF THE INVENTION

While illustrative embodiments of the invention have been described herein, the present invention is not limited to the various preferred embodiments described herein, but includes any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to examples described in the present specification or during the prosecution of the application, which examples are to be construed as non-exclusive. For example, in the present disclosure, the term “preferably” is non-exclusive and means “preferably, but not limited to.” In this disclosure and during the prosecution of this application, means-plus-function or step-plus-function limitations will only be employed where for a specific claim limitation all of the following conditions are present in that limitation: a) “means for” or “step for” is expressly recited; b) a corresponding function is expressly recited; and c) structure, material or acts that support that structure are not recited. In this disclosure and during the prosecution of this application, the terminology “present invention” or “invention” may be used as a reference to one or more aspect within the present disclosure. The language present invention or invention should not be improperly interpreted as an identification of criticality, should not be improperly interpreted as applying across all aspects or embodiments (i.e., it should be understood that the present invention has a number of aspects and embodiments), and should not be improperly interpreted as limiting the scope of the application or claims. In this disclosure and during the prosecution of this application, the terminology “embodiment” can be used to describe any aspect, feature, process or step, any combination thereof, and/or any portion thereof, etc. In some examples, various embodiments may include overlapping features. In this disclosure, the following abbreviated terminology may be employed: “e.g.” which means “for example;” and “NB” which means “note well.”

What is claimed is:

1. A method for the assessment of a joint, comprising: measuring musculoskeletal dynamics of a joint of a patient with the patient in a posture that is weight-bearing through said joint.

2. The method of claim 1, wherein said measuring musculoskeletal dynamics of a joint involves measuring musculoskeletal dynamics of an ankle.

3. The method of claim 2, wherein said measuring involves measuring musculoskeletal dynamics of an ankle in an inversion and/or eversion direction of rotation.

4. The method of claim 2, wherein said measuring musculoskeletal dynamics includes measuring inertia.

5. The method of claim 2, wherein said measuring musculoskeletal dynamics includes measuring damping.

6. The method of claim 5, wherein said measuring musculoskeletal dynamics includes measuring neuro-mechanical resistance to rotational velocity.

7. The method of claim 2, wherein said measuring musculoskeletal dynamics includes measuring stiffness.

8. The method of claim 7, wherein said measuring musculoskeletal dynamics includes measuring neuro-mechanical resistance to ankle rotational angle.

9. The method of claim 1, wherein said measuring musculoskeletal dynamics includes applying at least one pulse so as to excite a natural frequency at said joint.

10. The method of claim 9, where said measuring musculoskeletal dynamics includes acquiring position and torque sensor data after initiating said at least one pulse.

11. The method of claim 10, wherein said measuring musculoskeletal dynamics includes determining values of I, b or k based on the position and torque sensor data in accordance with the following formula:
{−Iω²+ibω+k}Θ(ω)=T(ω)
where I is inertia, b is resistance, k is stiffness, Θ is angular position, T is torque, ω is frequency, and i={−1}^{1/2}.

12. The method of claim 1, wherein said measuring musculoskeletal dynamics is performed using:
a) a platform to support the patient in a weight-bearing posture;
b) a drive mechanism to impart pulse movement to said platform; and
c) a displacement sensor to sense displacement of said platform.

13. The method of claim 12, wherein said measuring dynamics is also performed using:
a) a force or torque sensor to sense a force or torque on said platform;
b) a control system to determine musculoskeletal dynamics based on outputs from said displacement sensor and said force or torque sensor.

14. The method of claim 1, further including having said posture include standing.

15. The method of claim 1, further including having said posture include muscles at said joint being active during assessment.

16. A device for assessing musculoskeletal dynamics of a joint of a patient in a weight-bearing posture, comprising:
a) a platform to support a patient in a weight-bearing posture;
b) a drive mechanism to impart pulse movement to said platform;
c) a position sensor to sense a position of said platform;
d) a force or torque sensor to sense a force or torque on said platform;
e) a control system to determine musculoskeletal dynamics based on outputs from said displacement sensor and said force or torque sensor.

17. The device of claim 16, wherein said musculoskeletal dynamics includes inertia.

18. The device of claim 16, wherein said musculoskeletal dynamics includes damping.

19. The device of claim 16, wherein said musculoskeletal dynamics includes neuro-mechanical resistance to rotational velocity.

20. The device of claim 16, wherein said musculoskeletal dynamics includes stiffness.

21. The device of claim 16, wherein said musculoskeletal dynamics includes neuro-mechanical resistance to ankle rotational angle.

22. The device of claim 16, wherein said control system includes a control device configured to determine values of I, b or k based on data from the position sensor and the force or torque sensor in accordance with the following formula:
{−Iω²+ibω+k}Θ(ω)=T(ω)
where I is inertia, b is resistance, k is stiffness, Θ is angular position, T is torque, ω is frequency, and i={−1}^{1/2}.

23. The device of claim 22, wherein said control device includes a computer programmed to determine values of I, b or k based on data from the position sensor and the force or torque sensor.

24. The device of claim 16, wherein said control system includes a computer programmed to determine values of inertia, resistance and stiffness based on data from the position sensor and the force or torque sensor.

25. The device of claim 16, wherein said drive mechanism is controlled to impart a random or pseudorandom series of pulses.

26. The device of claim 25, wherein said drive mechanism includes an electric motor.

27. The device of claim 25, wherein said drive mechanism is controlled via a control device.

28. The device of claim 27, wherein said control device includes a programmed computer.

29. The device of claim 27, wherein a control device of the control system for determining musculoskeletal dynamics is independent from said control device that controls said drive mechanism.

30. The device of claim 27, wherein a control device of the control system for determining musculoskeletal dynamics is the same as said control device that controls said drive mechanism.

31. A device for assessing musculoskeletal dynamics of a body part of a patient in a weight-bearing posture, comprising:
a) a platform that supports a patient in a weight-bearing posture;
b) said platform being rotatably supported to rotate around an axis through said body part;
c) a drive mechanism that imparts a plurality of pulses to said platform at durations of less than about 50 milliseconds and at intervals of less than about 100 milliseconds;
d) an angle sensor that senses an angular position of said platform;
e) a torque sensor that senses a torque at said platform;
f) digital data storage having angle and torque time-based data from said angle and torque sensors.

32. The device of claim 31, further including at least one control system to control said drive mechanism and to determining musculoskeletal dynamics based on said data in said digital data storage.

33. The device of claim 31, wherein said device is configured to assess musculoskeletal dynamics of a patient's ankle in a weight-bearing upright standing posture and with the ankle rotating in an inversion and/or eversion direction upon said platform.

34. The device of claim 31, wherein said angle and torque time-based data obtained from said angle and torque sensors includes at least about 10 data points per second.

35. The device of claim 31, wherein said angle and torque time-based data obtained from said angle and torque sensors includes at least about 20 data points per second.

36. The device of claim 31, wherein said angle and torque time-based data obtained from said angle and torque sensors includes at least about 100 data points per second.
